(19) United States
(12) Patent Application Publication
(10) Pub. No.: US 2016/0038770 A1
(43) Pub. Date: Feb. 11, 2016

(54) FOCUSED TRANSCRANIAL ULTRASOUND SYSTEMS AND METHODS FOR USING THEM

(71) Applicant: THYNC, INC., Los Gatos, CA (US)

(72) Inventors: William J. TYLER, Newton, MA (US); Tomokazu SATO, Pasadena, CA (US); Alexander OPITZ, Goettingen (DE)

(21) Appl. No.: 14/781,459

(22) PCT Filed: Apr. 25, 2014
(86) PCT No.: PCT/US14/35413

§ 371 Date: Sep. 30, 2015

Related U.S. Application Data
(60) Provisional application No. 61/816,680, filed on Apr. 26, 2013.

(51) Int. Cl. A61N 7/00

(52) U.S. Cl. CPC A61N 7/00; A61N 2007/0026; A61N 2007/006; A61N 2007/0065

(57) ABSTRACT

Apparatus and methods for focusing transcranial ultrasound. The systems described herein are advantageous for noninvasive neuromodulation and other transcranial ultrasound applications such as high intensity focused ultrasound (HIFU). In particular, described herein are compound acoustic lens apparatus having a short focal length for use with a transcranial ultrasound system, systems including methods of using them. These compound lens assemblies allow transcranial stimulation of even superficial cortical regions of the brain for ultrasound neuromodulation with a compact, single transducer element system at low (e.g., 0.2 to 1 MHz) frequencies with relatively large diameter (e.g., >15 mm) transducers applying 1 to 10 watts/cm2 of acoustic energy (spatial-peak, temporal-average intensity at the target brain region), and short focal length (e.g., between 15 and 35 mm).

### CROSS REFERENCE TO RELATED APPLICATION

This patent application claims priority to the following provisional patent application which is herein incorporated by reference in its entirety: U.S. provisional patent No. 61/816,680, filed on Apr. 26, 2013, and titled “FOCUSED TRANSCRANIAL ULTRASOUND”.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

Described herein are apparatus (systems and devices) for the application of focused ultrasound to deliver transcranial ultrasound. The specification also relates to transcranial methods of using the focused ultrasound system, including for transcranial neuromodulation.

### BACKGROUND OF THE INVENTION

Recent research and disclosures have described the use of transcranial ultrasound neuromodulation to activate, inhibit, or modulate neuronal activity.

Noninvasive and nondestructive transcranial ultrasound techniques are in contrast to other transcranial ultrasound based techniques that use a combination of parameters to disrupt, damage, destroy, or otherwise affect neuronal cell populations so that they do not function properly and/or cause heating to damage or ablate tissue.

Ultrasound (US) has been used for many medical applications, and is generally known as cyclic sound pressure with a frequency greater than the upper limit of human hearing. An important benefit of ultrasound therapy is its non-invasive nature. US waveforms can be defined by their acoustic frequency, intensity, waveform duration, and other parameters that vary the timecourse of acoustic waves in a target tissue.

In diagnostic medical imaging, US is used in a frequency range from about 1 to 15 MHz, while therapeutic applications of US typically employ a frequency of about 1 MHz or less. As a fundamental property of its wave physics, higher acoustic frequencies of US confer greater spatial resolutions due to their shorter wavelengths. While lower acoustic frequencies of US have longer wavelengths and thus lower spatial resolutions, they are not as prone to scattering and can be transmitted longer distances with less attenuation compared to higher frequencies. Transducers for US imaging are designed to be highly sensitive transmit/receive devices, which transmit US into tissue then respond to the subsequent sound wave reflections off these tissues. Their high sensitivity enables them to respond to reflected waves having intensities only a fraction of the incident wave. Given such a high receive sensitivity, transcranial imaging applications can tolerate the high insertion loss of US which occurs at acoustic frequencies >1 MHz. Some therapeutic applications of US require power levels higher than those used for diagnostic imaging, but they typically do not require the submillimeter spatial resolutions conferred by high acoustic frequencies. Thus, the choice of US frequency used in a particular application must balance the needs for spatial resolution and power with the scattering, absorption, and transmission coefficients of tissues along the beam path.

To affect brain function transcranial ultrasound neuromodulation requires appropriate ultrasound waveform parameters, including acoustic frequencies generally less than about 10 MHz, spatial-peak temporal-average intensity generally less than about 10 W/cm² (e.g., between 0.5 and 10 W/cm²), and appropriate pulsing and other waveform characteristics to ensure that heating of a targeted brain region does not exceed about 2 degrees Celsius for more than about 5 seconds. Transcranial ultrasound neuromodulation induces neuromodulation primarily through vibrational or mechanical mechanisms. Noninvasive and nondestructive transcranial ultrasound neuromodulation is in contrast to other transcranial ultrasound based techniques that use a combination of parameters to disrupt, damage, destroy, or otherwise affect neuronal cell populations so that they do not function properly and/or cause heating to damage or ablate tissue. A patent and patent applications from one of the named inventors describe systems and methods for transcranial ultrasound neuromodulation, including: U.S. Pat. No. 8,591,419 and PCT application US2009/050560 titled “Methods and systems for transcranial ultrasound neuromodulation” by inventor Tyler; and PCT application US2010/055527 titled “Devices and methods for modulating brain activity” by inventor Tyler.

Phased arrays of ultrasound transducers are well-known as a system for focusing ultrasound energy at target sites inside the body. Constructive and destructive interference of acoustic waves transmitted by multiple transducers can be used to deliver complex spatiotemporal patterns of acoustic waves. Generally, phased arrays use tens to hundreds or even thousands of ultrasound transducers distributed spatially on the surface of the body. For instance, a phased array placed on the head can be used to target an area deep in the brain. However, phased arrays have important limitations for delivering ultrasound transcranially for neuromodulation. Phased arrays use spatially distributed transducers, requiring a larger form factor. Moreover, large and generally unportable power and control components are required to manage the timing, intensity, phase, and other properties of the ultrasound waves transmitted by each of the transducers.

Another common technique for focusing ultrasound is by using a shaped lens with an acoustic velocity (i.e. speed of sound) that differs from adjoining air, tissue, or material to bend acoustic waves. Most standard ultrasound focusing lenses employ a single concave lens. However, a single concave lens focusing system for ultrasound has limitations, including limitations related to miniaturization and portability. Ultrasound lenses comprised of a single concave lens necessarily require a transducer assembly form factor that extends further axially (perpendicular to the skull) due to the required thickness of the lens. Ultrasound lenses comprised of a single concave lens are also limited with regard to the range of focal lengths that can be achieved with a lens of a particular cross sectional area. Short focal lengths cannot be achieved with smaller cross sectional areas appropriate for systems affixed to the head or skull. A good analogy is with glass lenses for optics, where focusing lenses have limits on how short a focal length they can have for a given diameter. Neuromodulation of superficial brain regions with an appropriate transcranial ultrasound system would be advantageous due to the importance of such superficial brain regions (e.g. cerebral cortex) to sensory, motor, higher cognitive function, and other brain functions.

New systems and methods for focusing ultrasound energy transcranially would be beneficial for transcranial ultrasound neuromodulation and other transcranial ultrasound applications. An advantageous feature of new systems and methods for focusing ultrasound energy transcranially would be smaller and more energy efficient systems and those that can effectively focus at superficial targets in the brain. These and other features and advantages of the present invention will be explained and will become understood to one skilled in the art through the summary of the invention that follows.

### SUMMARY OF THE INVENTION

Described herein are apparatus (including devices and systems) for focusing transcranial ultrasound, as well as methods of using these apparatus to apply transcranial neuromodulation by ultrasound. The apparatus described herein may be advantageous for noninvasive neuromodulation and also for other transcranial ultrasound applications such as high intensity focused ultrasound (HIFU) for thermal ablation, as well as other applications. Compound (e.g., convex-concave) lens assemblies described herein (“VexCave lenses”) have beneficial properties for focusing ultrasound into the body including transcranially into the brain for ultrasound neuromodulation.

The transcranial ultrasound neuromodulation focusing apparatus and methods described herein may use a compound lens assembly to achieve tighter focusing and shorter focal lengths relative to a single concave lens system.

A beneficial aspect of the compound concave-convex ultrasound transcranial ultrasound neuromodulation focusing systems and methods described herein is miniaturization, permitting transcranial ultrasound focusing with a compact, single transducer element system.

The apparatus and methods described herein may provide energy transmission similar to that provided by more complex and expensive ultrasound phased arrays, at lower (more shallow) focal depths. In particular, the apparatus and methods described herein may be used with a single ultrasound transducer in a manner that does not require the expensive and complex drive circuitry necessary for phased arrays, and may instead be used with a single drive element and transducer. However, the principles described herein, including the compound lenses may be adapted for use in a phased array.

In general, the apparatus and methods described herein may allow focusing of the ultrasound energy with a short focal length (e.g., less than twice the diameter of the transducer). This short focal length is not typically possible with standard devices adapted to operate at low frequency and appropriate intensity for neuromodulation (e.g., between 0.2 and 1 MHz and intensity of about 0.5-10 watts/cm²). For example, a typical concave lens would not be able to get the field within this short focal length, as may be particularly important when applying neuromodulatory ultrasound stimulation to cortical regions (e.g., near the skull). Notwithstanding the advantages of the apparatus and methods described herein for focusing with a short focal length, the VexCave lenses described herein can be adapted to have longer focal lengths.

For example, the apparatus and methods described herein may be used to target tissue within about 10-35 mm of the apparatus when applied to a head (e.g., within 15-30 mm, within 20-25 mm, etc.) to target cortical regions. In general, the focal length for low frequency neuromodulation using standard transducers and focusing has been estimated as greater than twice the diameter of the transducer.

Further, it is beneficial in many configurations to use transducers that are no smaller than some minimum diameter (e.g., 15 mm, 16 mm, 17 mm, 18 mm, 19 mm, 20 mm, 21 mm, 22 mm. 23 mm, 24 mm, 25 mm, etc.), as the material properties of the transducer may limit the minimum size at the desired frequency and intensity ranges; in addition, the diameter of the transducer may also correspond to aperture size. In some uses, it may be beneficial, particularly when stimulating neuronal tissue networks to neuromodulate, to have a stimulation region (size, e.g., spot size) that is sufficiently large, even while having a shallow penetration depth from a transducer, allowing the operator to modulate a larger area.

The transducers and compound acoustic lenses described herein may provide highly focused ultrasound stimulation while minimizing standing waves. Thus, when the focus is near the diffraction limit in the tissue (staying within the near field), such as focusing to within approximately 1.5× diffraction limit, standing waves may be avoided using the compound lenses described herein.

In general, the apparatus and methods describe herein provide a shorter focal distance for transducers having relatively large diameter and a given thickness by forming a “thin” compound lens in which a concave lens partially encloses a convex lens, which helps further shorten the focus. In particular, the apparatuses and methods described herein provide acoustic systems including compound acoustic lenses that have a high numerical aperture; these lenses have a higher numerical aperture than could otherwise be possible for comparable single lens systems. These benefits are enabled, in part, by the use of two (or more lenses) forming the compound lens in which the speed of sound in a concave lens is greater than the speed of sound in water (e.g., biological media), which is greater than the speed of sound in a convex lens. As described in greater detail below, this may help determine the focusing for the compound lens system. In addition, the compound lens may be adapted to minimize or lessen reflective losses. For example, the acoustic impedance of the concave lens to be placed closest (e.g., adjacent to) the transducer may be lower than the acoustic impedance of the transducer. Further the acoustic impedance of subsequent elements before contacting the subject may be successively lower until they are near (or match) the impedance of the biological tissue to which the acoustic energy is applied. For example, the acoustic impedance of the convex lens adjacent to the concave lens (opposite the transducer) may be lower than the acoustic impedance of the concave lens. This may lessen reflective losses. In building the compound lenses described herein, the choice of materials forming the lenses may be based primarily on the speed of sound through the materials and their geometries (e.g., focusing) rather than the impedances, so, for example, the acoustic impedance of the transducer may be slightly less than the acoustic impedance of the concave lens, and/or the acoustic impedance of the convex lens may be slightly less than the impedance of water (e.g., tissue).

For example, described herein are compound acoustic lens apparatus having a short focal length for use with a transcranial ultrasound system. Any of these apparatus may include: an ultrasound transducer having a diameter (or may be adapted for connection to a transducer having a diameter); a concave lens coupled to the ultrasound transducer, wherein the concave lens has an acoustic velocity that is greater than an acoustic velocity of water; and a convex lens coupled to the concave lens, wherein the convex lens has an acoustic velocity that is less than the acoustic velocity of water; further wherein the focal length of the compound acoustic lens is 1.5 times the diameter of the ultrasound transducer or less when a frequency of acoustic energy applied from the compound acoustic lens is between about 0.2 MHz and 1 MHz at a spatial-peak, temporal-average intensity of about 10 watts/cm² or less. Typically, the diameter of the ultrasound transducer is 15 mm or greater.

A compound acoustic lens apparatus having a short focal length for use with a transcranial ultrasound system may include: an ultrasound transducer having a diameter of about 15 mm or greater (or the compound lens may be configured or adapted to operate with such a transducer); a concave lens coupled to the ultrasound transducer, wherein the concave lens has an acoustic velocity that is greater than an acoustic velocity of water; and a convex lens coupled to the concave lens, wherein the convex lens has an acoustic velocity that is less than the acoustic velocity of water; wherein the focal length of the compound acoustic lens is less than twice the diameter of the ultrasound transducer when a frequency of acoustic energy applied though the compound acoustic lens is between about 0.2 MHz and 1 MHz at a spatial-peak, temporal-average intensity of about 10 watts/cm² or less.

The focal length of any of the compound acoustic lenses described herein may be about 1.5 times the diameter of the ultrasound transducer (e.g., about 1.4 time the diameter, about 1.3 times the diameter, about 1.2 times the diameter, about 1.1 times the diameter, about the same as the diameter, etc.) or less when a frequency of acoustic energy applied from the compound acoustic lens is between about 0.2 MHz and 1 MHz at a spatial-peak, temporal-average intensity (I_{SPTA}) of between about 0.5 and about 10 watts/cm² at a target brain region. Alternatively or additionally, the focal length of any of the compound acoustic lenses described herein may be about 1.9 (or 1.8, 1.7, 1.6, 1.5, 1.4, 1.3, 1.2, 1.1, 1.0, etc.) times the diameter (or less) of the ultrasound transducer when a frequency of acoustic energy applied from the compound acoustic lens is between about 0.2 MHz and 1 MHz at a spatial peak, peak average intensity (I_{SPPA}) of less than about 300 W/cm² (e.g., 250 W/cm², 200 W/cm², 190 W/cm², 180 W/cm², 170 W/cm², 150 W/cm², 100 W/cm², 50 W/cm², etc.), e.g., at a target brain region.

In general, the concave lens of the compound lens may be positioned between the transducer and the convex lens, and may be immediately adjacent a face of the ultrasound transducer with the convex lens immediately adjacent the concave lens.

Any appropriate convex and concave lens may be used. For example, the convex lens may be a plano-convex lens, e.g., having a convex surface facing away from the transducer. The concave lens may be, for example, a plano-concave lens having a concave surface facing the transducer.

Any of the compound lenses described herein may include only the concave and nested convex lens, or they may also include one more additional lenses, or may be used with one or more additional lenses, such as: polymeric; bi-concave; bi-convex; plano-concave/plano-convex; fixed radius, parabolic, hyperbolic, cylindrical lens shapes; Fresnel lens, or like.

As mentioned above an apparatus including the compound lens may also be configured to reduce reflective losses. For example, an acoustic impedance of the transducer may be greater than an acoustic impedance of the concave lens, and the acoustic impedance of the concave lens may be greater than an acoustic impedance of the convex lens.

The compound lenses described herein may have a short focal length even while having a relatively large diameter of the transducer used. For example, the focal length of the compound lens may be less than 35 mm (e.g., when the diameter, D, of the transducer has a diameter that is 15 mm or greater (e.g., 16 mm or greater, than 17 mm or greater, 18 mm or greater, than 19 mm or greater, than 20 mm or greater, 21 mm or greater, 22 mm or greater, than 23 mm or greater, 24 mm or greater, 25 mm or greater, etc.)).

The concave lens forming any of the compound lenses described herein may be formed of (e.g., primarily of or entirely of) a material selected from the group consisting of: graphite or aluminum. Any appropriate material may be used, such as materials in which the speed of sound through the material is greater than that of water (e.g., depending on temperature, between about 1400-1543 m/sec). When describing or including the speed of sound (and/or the relative speeds of sound) of materials, the temperature may be assumed, unless the context specifies otherwise to be body temperature (e.g., 37° C.) or the temperature of the target material. For example, the speed of sound of materials that may be used to form the concave lens of the compound lenses described herein such as aluminum (approximately 6420 m/s) or graphite (approximately 5950 m/sec) is typically much faster than the speed of sound in water (approximately 1433 m/sec). The speed of sound through materials that may be used to form the convex lens of the compound lens, such as rubbers (approximately 40-150 m/sec), balsa (approximately 800 m/s) and cork (approximately 366-518 m/sec) are generally at least slightly less than the speed of sound through water (e.g., 1433 m/sec). Some polymeric materials (silicones, polycarbonates, polyurethane) and other materials (e.g., Teflon) may have speeds of sounds that are less than water. The material forming the lenses may be oriented so that the speed of sound through the lens is based on the speed through the orientation in which the sound energy is passing (e.g., parallel to the lens, transverse to the lens, etc.).

Also described herein are systems for neuromodulation by transcranial ultrasound, the system comprising: an ultrasound transducer having a diameter; a compound acoustic lens having a short focal length, the apparatus comprising: a concave lens coupled to the ultrasound transducer, wherein the concave lens has an acoustic velocity that is greater than an acoustic velocity of water, and a convex lens coupled to the concave lens, wherein the convex lens has an acoustic velocity that is less than the acoustic velocity of water, wherein the focal length of the compound acoustic lens is less than 1.5 times the diameter of the ultrasound transducer during operation of the system; and a driver coupled to the ultrasound transducer and configured to drive the ultrasound transducer to emit a frequency of acoustic energy from the compound acoustic lens between about 0.2 MHz and 1 MHz at a spatial-peak, temporal-average intensity of about 10 watts/cm² or less.

Any of the compound acoustic lenses described herein may be included as part of the systems described.

In some cases it may be useful to provide methods and apparatuses (e.g., systems) in which more than one compound lens may be used, for example, systems or apparatuses in which the transducer, or a separate transducer, is coupled to compound lenses having other focal properties, including different focal lengths.

For example, a system (or method of operating a system) may include different compound acoustic lenses that can be swapped in and out of a transducer assembly in order to target different depths, much as one would do for different objectives fitting on a microscope. In the context of stimulation such as neurostimulation, different compound acoustic lenses may be used to treat tissue at different depths and/or treatment volumes. For example, any of the acoustic compound lenses described herein may be swappable lenses that may be interchanged with a single transducer and/or system. For example, an ultrasound transducer base may be used with multiple, swappable compound lenses. In some variations, the transducer may be swapped with the compound lens (e.g., the compound lens may include the transducer).

For example, a system may include a transducer that has an adjustable focal length that is adjustable in increments of 0.5 mm or so. Alternatively or additionally, the focal length may stay the same between different (swappable) compound lenses, but the shape of the acoustic field may be changed based on static or active elements within the compound lens. In some variations the compound acoustic lens may include a smart fluid, e.g., forming an intermediate layer, that could modulate the acoustic beam through the compound lens. Either way just changing lens elements which are coupled to the face of the transducer using a thin replaceable silicon layer would be a beneficial feature in a system. A smart fluid may refer to a fluid whose properties (including, for example, acoustic properties) can be changed by applying an electric field or a magnetic field.

Also described herein are methods for transcranial neuromodulation by applying transcranial ultrasound using a compound acoustic lens having a short focal length, the method comprising: driving an ultrasound transducer having a diameter, to emit a frequency of acoustic energy from between about 0.2 MHz and 1 MHz; passing the acoustic energy through a concave lens of a compound acoustic lens that is attached to the ultrasound transducer, wherein the concave lens has an acoustic velocity that is greater than an acoustic velocity of water; passing the acoustic energy from the concave lens through a convex lens of the compound acoustic lens, wherein the convex acoustic lens has an acoustic velocity that is less than the acoustic velocity of water; and focusing the acoustic energy leaving the compound acoustic lens at a focal length of less than 1.5 times the diameter of the ultrasound transducer to target a brain region and deliver the acoustic energy at a spatial-peak, temporal-average intensity of about 10 watts/cm² or less to the target brain region.

In general, these methods may be applied to neuromodulate a subject by applying the transducer and compound lens either directly or through an intermediary (e.g., pad, gel, etc.) to the subject's head directed to a region of the subject's brain, including superficial cortical regions. For example, the step of focusing may comprise non-invasively focusing the acoustic energy on a cortical region of a brain. Thus, any of these methods may also include positioning the ultrasound transducer and compound acoustic lens against a subject's head.

As mentioned, the methods may include positioning the ultrasound transducer and the compound acoustic lens against an intermediary, such as a gel or solid ultrasound couplant placed against a subject's head. The couplant/intermediary may be a silicone gel (e.g., pad, cover, etc.). For example, the method may include positioning the ultrasound transducer and the compound acoustic lens against an ultrasound couplant comprising silicone that is placed against a subject's head.

In general, passing the acoustic energy through the concave lens may comprise passing the acoustic energy through the concave lens that is positioned adjacent to a face of the ultrasound transducer on one side and is positioned adjacent to the convex lens on the opposite side. The step of passing the acoustic energy from the concave lens through a convex lens may comprise passing the acoustic energy from the convex lens wherein the convex lens is a plano-convex lens having a convex surface facing the transducer. Passing the acoustic energy through the concave lens may comprise passing the acoustic energy through the concave lens wherein the concave lens is a plano-concave lens having a concave surface facing away from the transducer.

In general, focusing the acoustic energy leaving the compound acoustic lens may therefore comprise focusing the acoustic energy at a focal length of less than 35 mm (e.g., less than 34 mm, less than 33 mm, less than 32 mm, less than 31, less than 30 mm, less than 29 mm, less than 28 mm, less than 27 mm, less than 26 mm, less than 25 mm, etc., including between about 10 and about 35 mm, between about 15 and about 30 mm, between about 15 and about 25 mm, etc.).

### BRIEF DESCRIPTION OF THE DRAWINGS

The invention has other advantages and features which will be more readily apparent from the following detailed description of the invention and the appended claims, when taken in conjunction with the accompanying drawings, in which:

FIG. 1: Schematic showing a transcranial ultrasound focusing system in accordance with an embodiment of the present invention.

FIGS. 2A-2C illustrate an exemplar ultrasound transducer and compound convex-concave acoustic lens assembly in exploded side, top perspective, and alternate top perspective views, respectively. FIG. 2D shows a front and back view of an ultrasound transducer with attached compound acoustic lens for transcranial applications.

FIGS. 3A-3B show acoustic pressure and transmission (axial distance) relationships at various frequencies (FIG. 3A) and compared to theoretical models (FIG. 3B).

FIGS. 4A-4D illustrate quantitative acoustic field mapping of focused ultrasound in accordance with an embodiment of the present invention.

FIGS. 5A-5D show quantitative acoustic field mapping of focused ultrasound in accordance with an embodiment of the present invention.

FIGS. 6A-6D show data plots showing results of quantitative acoustic field mapping for lateral and axial cross sections.

FIGS. 7A-7F show modeled acoustic intensity delivered transcranially targeting dorsolateral prefrontal cortex in accordance with an embodiment of the present invention.

FIGS. 8A-8F show modeled acoustic intensity delivered transcranially targeting primary motor cortex in accordance with an embodiment of the present invention.

FIGS. 9A-9F show modeled acoustic intensity delivered transcranially targeting the occipital cortex in accordance with an embodiment of the present invention.

FIG. 10A shows intensity of applied acoustic energy from an ultrasound transducer coupled to a compound acoustic lens as described herein. FIG. 10B shows a model (finite element model) of a human head to which the same transducer and compound acoustic lens having a short focal length is applying acoustic energy.

FIGS. 11A and 11B illustrate the difference in the focal depth between a transducer with a compound acoustic lens as described herein (FIG. 11A) and the same transducer with a typical single concave lens (FIG. 11B). The focal length of the compound lens is much shorter than the concave-only lens.

### DETAILED DESCRIPTION OF THE INVENTION

Described herein are transcranial ultrasound focusing systems and methods for the use thereof. In some embodiments, transcranial ultrasound focusing systems as described herein are configured to modulate brain function. The systems and methods described herein may be advantageous for noninvasive neuromodulation and permit focusing ultrasound energy transcranially at one or more target regions, including superficial target regions such as cerebral cortex.

Transcranial ultrasound neuromodulation is useful for affecting brain function by activating, inhibiting, or modulating neuronal activity. According to an embodiment of the present invention, a transcranial ultrasound neuromodulation system comprises at least one ultrasound transducer and appropriate hardware and/or software (e.g. firmware) for controlling the intensity, duration, pulsing, acoustic frequency, and other parameters of ultrasound energy delivered. The controlling hardware, software, and/or firmware may be incorporated into a controller (e.g. including one or more processors) and may receive control inputs from a user or other operator, controlling operation of such systems. In some embodiments of the invention, a transcranial ultrasound system incorporates one or more features selected from the group consisting of: self-coupled (i.e. incorporating an acoustic couplant material to form a low acoustic impedance contact with the head), a solid acoustic couplant, and one or more capacitive micromachined ultrasound transducers (CMUTs).

For transcranial ultrasound applications, shorter focal lengths are advantageous for targeting cortical and other regions near the brain surface. For portable and other miniaturized transcranial ultrasound applications (e.g. in contrast to large ultrasound arrays that require complex and large control circuitry), smaller acoustic lens and transducer assembly systems are advantageous. Transducer assemblies that incorporate compound convex-concave lenses in accordance with description provided herein may achieve shorter focal lengths with form factors that are smaller axially and cross-sectionally. In some embodiments, transducer assemblies incorporating compound convex-concave lenses are comprised of a single transducer element. In other embodiments, the transducer assembly comprises multiple transducer elements, wherein the multiple transducer elements can be optionally configured as an array of ultrasound transducers. The compound lenses described herein may be integrated onto the ultrasound transducer or may be adapted to be placed on/taken off of an ultrasound transducer.

The apparatus (e.g., systems and devices) and methods described herein may provide short focal length lenses for focusing ultrasound transcranially. Effective focusing is advantageous for transcranial ultrasound neuromodulation. By focusing ultrasound energy at one or more targeted brain regions, less total ultrasound energy needs to be delivered to achieve neuromodulation at the targeted site, reducing power consumption as well as heating of components of the ultrasound transducer assembly, focusing assembly, and coupling assembly. Another advantage of effective focused transcranial ultrasound neuromodulation is to reduce or eliminate unwanted neuromodulation in brain regions proximal, distal, or nearby the targeted region, as well as to reduce heating of non-target biological tissue for the same peak spatial power.

A transducer assembly configured with a compound convex-concave lens for transcranial applications enables miniaturization and provides the ability to focus transcranially at superficial brain regions. Thus, the ultrasound transducer systems described herein can be used for a variety of applications, including but not limited to transcranial neuromodulation, high frequency ultrasound (e.g., containing a dominant acoustic frequency >1 MHz, greater than 5 MHz, greater than 10 MHz, greater than 15 MHz, etc.), high intensity ultrasound (i.e. delivering power >1 W/cm² at the site of the target brain tissue (and in some applications, causing heating and/or ablation)), and other applications of transcranial ultrasound for imaging, therapy, ablation, and neuromodulation (including neuromodulation of peripheral targets such as peripheral nerves and spinal cord).

A compound acoustic lens as described herein may include a convex lens and a concave lens to focus and couple ultrasound energy from an ultrasound transducer to the head of a subject for delivering ultrasound energy transcranially. Various ultrasound transducers can be used to generate the acoustic wave. Specific water immersion type transducers are the Ultran GS500-D13, NDT Systems IBMF0.53, Ultran GS350-D19, Olympus Panametrics V318 focused transducer 0.5 MHz/0.75″ F=0.85″, Ultran GS200-D25 and Olympus Panametrics V301S 0.5 MHz/1.0″. Non-water immersion type transducers may be used for systems incorporating a compound convex-concave lens in which a concave lens is coupled directly to the ultrasound transducer element. There are numerous types of ultrasound transducers that could be used with embodiments of the present invention, and embodiments of the present invention are contemplated for use with any appropriate ultrasound transducer.

A compound convex-concave lens assembly may have a concave lens that is positioned more proximal to the transducer and a convex lens distal to the concave lens relative to the transducer assembly. The two lenses are typically fixed in place relative to each other and one or both of the lenses may be configured to be self-adhering to one or more other components of the system. In various embodiments, the lenses are fixed with a glue, epoxy, or via a mechanical assembly such as a housing or coupling piece. Any appropriate methods for affixing the lenses in place may be used.

Compound lenses comprised of more than two lenses may be used for delivering focused transcranial ultrasound. For example, a compound acoustic lens may also include one or more lenses in addition to the concave (piano-concave or bi-concave) and convex (plano-convex or bi-convex) lenses, such as: polymeric; bi-concave; bi-convex; plano-concave/plano-convex; fixed radius, parabolic, hyperbolic, cylindrical lens shapes; Fresnel lens, or like.

Acoustic focusing by the compound convex-concave acoustic lenses described herein may be achieved by selecting lens materials based on the speed of sound through the material that comprises them. Specifically, the material comprising the convex lens (located further from the transducer than the concave lens) may have a speed of sound that is slower than the speed of sound in the target biological tissue (optionally estimated as the speed of sound in water). The material comprising the concave lens may have a speed of sound that is faster than the speed of sound in the target biological tissue (optionally estimated as the speed of sound in water).

Tables of acoustic properties of materials are well-known to one skilled in the art and can be used to select appropriate materials for each of the lenses. For instance, the Onda Corporation provides lists of acoustic properties for many materials (e.g., http://www.ondacorp.com/tecref_acoustictable.shtml). Materials for the lenses can be machined, molded, or otherwise shaped for desired acoustic focusing. Examples of materials with a speed of sound faster than water that may be used for the concave lens include graphite and aluminum. Examples of materials with a speed of sound slower than water that may be used for the convex lens include silicone rubbers (such Silgard 170, and RTB 11), balsa wood, and cork. The lenses may be formed so that the materials are oriented so as to provide the desired speed of sound, as the speed of sound through many solids may vary based on orientation of the material.

Acoustic focusing by the compound convex-concave lens can also be related to shape (morphology) of the lens, thus any of the lenses forming the compound acoustic lens may be shaped to achieve a desired focal distance. Ultrasound modeling software can be of great use in designing compound convex-concave lenses. Many academic groups write their own code as part of research that is distributed as freeware, as well known to one skilled in the art. An example of ultrasound modeling software is the Field II Stimulation program available from the Center for Fast Ultrasound Imaging in Denmark. Another example of ultrasound modeling software is PZ Flex available from Weidlinger Associates. Although the thickness of the concave and convex lenses may be flexible, there is typically only one degree of freedom for the radius of curvature and thus it is difficult to have independent distancing between consecutive lenses.

In general, the convex lens (with the lower speed of sound than target tissue/water) may be nested in the concavity of the concave lens (having a higher speed of sound that the target tissue/water), and the concave lens may be positioned adjacent to the transducer. The convex lens of the compound acoustic lens may be a plano-convex lens; the concave lens may be a plano-concave lens. The compound convex-concave acoustic lens of a transcranial focused ultrasound system may consist of a plano-concave lens proximal to the transducer and a plano-convex lens that fills the distal concavity of the plano-concave lens. In some embodiments, the distal plano-convex lens is designed to fit into the aperture of the plano-concave lens and is constructed of a material with a lower speed of sound such as silicone.

To improve the efficiency of transmission of acoustic energy from the ultrasound transducer through the compound convex-concave acoustic lens and into the subject's head, the compound acoustic lens may be designed with materials having appropriate relative acoustic impedance properties. As mentioned above, the speed of sound through the materials forming the lenses and their arrangements is of primary concern; secondarily the materials forming the lenses may be chosen to minimize loss. For example, compound convex-concave acoustic lens systems may select materials with appropriate acoustic impedance that improves the efficiency of ultrasound transmission and reduces heating. For example, effective ultrasound transmission occurs when the following criteria are met: the acoustic impedance of the ultrasound transducer is greater than the acoustic impedance of the material comprising the concave lens; the acoustic impedance of the material comprising the concave lens is greater than the acoustic impedance of the material comprising the convex lens; and the acoustic impedance of the material comprising the convex lens is greater than the acoustic impedance of the user's head (estimated to be equal to the acoustic impedance of water). While heat can affect the properties of these lenses, at the normal temperature ranges of operation, especially in biological applications, these changes can be mostly neglected.

Any of the ultrasound transducer systems provided herein may be used for focusing ultrasound transcranially through the skull of a subject into the dura, brain, and other tissue. For example, a transducer system may provide a portable and/or handheld unit that can be used in a variety of applications. In an embodiment, the transducer system includes one or more lenses for focusing ultrasound waves into the brain. In an embodiment the lenses are designed so that the resulting ultrasound is focused to a point at or near the surface of the brain when the transducer assembly is placed on a subject's head. In embodiments of the invention, the compound convex-concave acoustic lens and ultrasound transducer system are configured to achieve a focal distance greater than the distance between the face of the ultrasound transducer and the surface of the brain. Compound convex-concave lenses can be designed to target various depths, including superficial depths less than about 1 cm from the surface of the brain. Thus, the ultrasound transducer systems described herein, including the compound acoustic lenses described herein, may send ultrasound to superficial areas of the cerebral cortex near the surface of the brain that are generally not reachable by phased arrays of ultrasound transducers.

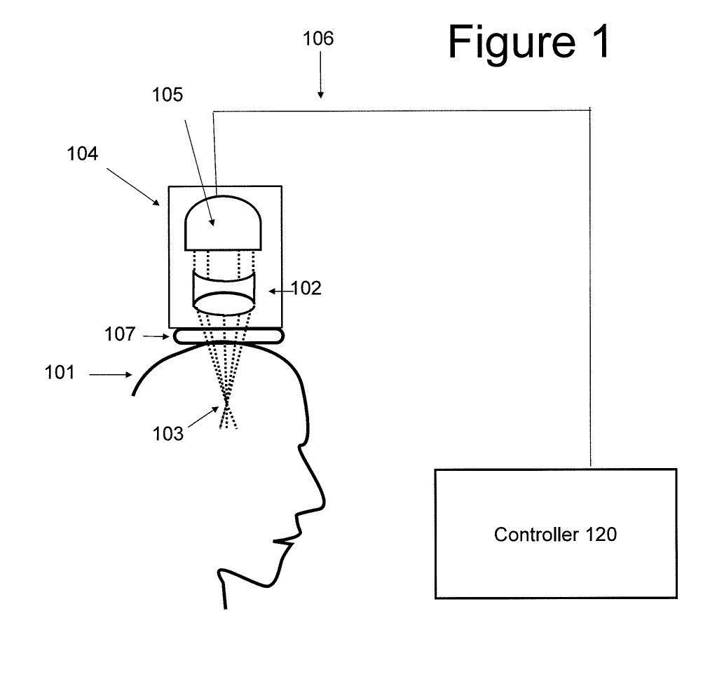

FIG. 1 shows a schematic of a transcranial ultrasound system that incorporates a compound convex-concave lens, in accordance with an embodiment of the present invention. Ultrasound controller assembly 120 provides control signals, power, and, in some embodiments, other communications, via wire assembly 106 to ultrasound transducer and compound convex-concave lens housing 104. In some embodiments, some or all components of the ultrasound controller assembly are contained in the same housing as the ultrasound transducer and compound convex-concave lens. Ultrasound transducer 105 is acoustically coupled to compound convex-concave acoustic lens 102. Housing 104 and/or the compound lens 102 may be acoustically coupled to head of user 101 directly (not shown) or with acoustic couplant 107 (e.g. ultrasound gel, or silicone pad) for efficient transmission of acoustic energy that is focused at targeted brain region 103. The total focal depth from the compound lens may be 35 mm or less (e.g., 30 mm or less, 25 mm or less, etc.).

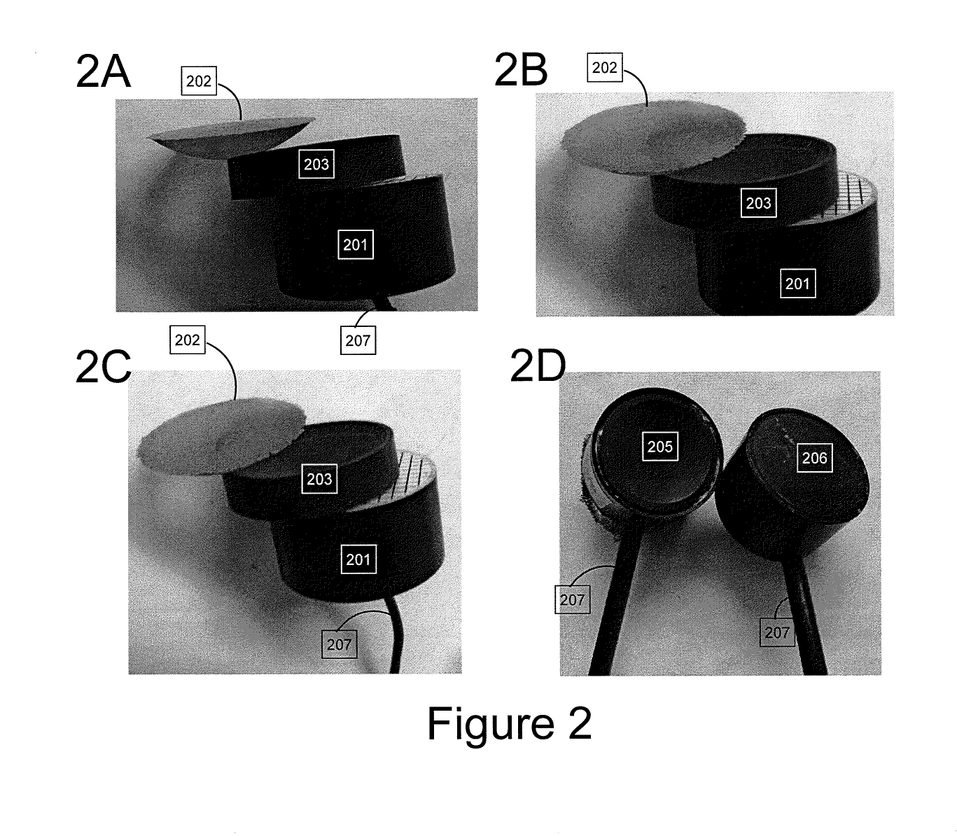

FIGS. 2A-D show a single-element ultrasound transducer (e.g., a single transducer) integrated with a compound convex-concave acoustic lens, as described herein. Suitable assemblies are available, for instance, from Blatek, Inc. (State College, Pa.). FIGS. 2A-2D show different views of two compound convex-concave lens and ultrasound transducer assemblies. FIGS. 2A, 2B, and 2C show different views of a disassembled compound convex-concave lens and ultrasound transducer assembly, including components: power and controller wire 207, ultrasound transducer 201, concave acoustic lens 203, and convex acoustic lens 202. A fully assembled unit is shown to the right in FIG. 2D at 206, and assembly 205 lacking the distal convex lens. Each of the compound convex-concave acoustic lens and ultrasound transducer assemblies shown are about 30 mm in diameter with a concave lens component having a thickness of about 6 mm and the convex lens component having a thickness of about 5.5 mm. The radius of curvature was estimated to be about 21.75 mm based on the diameter and thickness by assuming spherical curvature and using the equation: Radius of curvature=(Thicknesŝ2+Diameter̂2/4)/2/Thickness. There are numerous dimensions and shapes of the transducer assembly and lenses that can be selected to achieve a particular focus with a desired form factor and size while still applying the features of the compound acoustic lenses (e.g., short focal length, arrangement of concave and convex sub-lenses, material properties of the sub-lenses, etc.).

The concave lens of the compound convex-concave assembly may be in direct contact with the face of an ultrasound transducer or housing of an ultrasound transducer assembly, and may be positioned directly adjacent to the transducer. The convex lens of the compound convex-concave assembly may be in direct contact with the body (e.g. the head) of a subject.

The convex and concave lens components of the focused ultrasound transducer assembly may be held together with any appropriate material (e.g., epoxy or glue). Alternatively, the lenses can be held in physical contact through mechanical methods such as one or more components of the housing of the ultrasound transducer and lens assembly. If a bonding agent is used to hold the lenses together, it may be free of air bubbles or other aberration or gaps which can lead to significant reflections, losses, and aberrations in focusing. If the concave lens is made of a moldable material such as silicone, the bonding of the concave lens to the convex lens may be sufficiently strong to not require additional adhesive methods. Suitable ultrasound transducer and compound convex-concave lens materials are available, for instance, from Blatek, Inc. (State College, Pa.).

Typically, the degree of focus lies in a continuum, and the limits on diameter and thickness of the transducer, acoustic numerical aperture (NA) required (perhaps governed by heating properties), and depth of focus (depending on target depth) can all affect transducer design. In general, thicker systems may achieve more focusing since there is more time that the ultrasound wave front is traveling in media of different speed. However, if the system is too thick, the focal point of the lens system may be within the lens system rather than—as intended—to be within the brain at a target region. In some embodiments, a compound convex-concave lens of diameter D would have a thickness of at least D/8.

Efficient coupling from a compound convex-concave and ultrasound transducer assembly into the head can be achieved by using an appropriate ultrasound couplant (e.g., coupling gel, liquid, or solid couplant) as well known to one skilled in the art of ultrasound for biological applications.

As mentioned, a transcranial ultrasound assembly may incorporate an array of transducers in addition to one or more compound concave-convex focusing acoustic lens. Capacitive micromachined ultrasound transducers (CMUTs) are advantageous for creating ultrasound transducer arrays, because they can be manufactured inexpensively and at high density. In an embodiment of the invention, a CMUT array is configured to be a phased array to provide further focusing of ultrasound energy than is possible with a compound convex-concave lens alone.

The effect of transcranial ultrasound neuromodulation on brain function may be detected by one or more technique selected from the group that includes, but is not limited to: (i) subjectively by the recipient as a perception, movement, concept, instruction, other symbolic communication by modifying the recipient's cognitive, emotional, physiological, attentional, or other cognitive state; (ii) through physiological measurement of brain activity by one or a plurality of: electroencephalography (EEG), magnetoencephalography (MEG), functional magnetic resonance imaging (fMRI), functional near-infrared spectroscopy (fNIRS), positron emission tomography (PET), single-photon emission computed tomography (SPECT), computed tomography (CT), functional tissue pulsatility imaging (fTPI), and other techniques for measuring brain activity known to one skilled in the art; and (iii) by making a physiological measurement of the body such as by electromyogram (EMG), galvanic skin response (GSR), heart rate, blood pressure, respiration rate, pupil dilation, eye movement, gaze direction, and other physiological measurement. In an embodiment of the invention, the transcranial ultrasound neuromodulation assembly further comprises one or more appropriate sensors, transducers, electrical control circuitry, signal processing systems or any combination thereof, configured to achieve one or more of the above listed techniques for measuring the physiology or brain activity of the user.

A transcranial ultrasound neuromodulation protocol may deliver ultrasound to one or more brain regions and induces neuromodulation that correlates more strongly in time with the timecourse of mechanical effects on tissue than thermal effects. The acoustic frequency for transcranial ultrasound neuromodulation is generally greater than about 100 kHz and less than about 10 MHz, i.e. generally greater than about 100 kHz and less than about 10 MHz; optionally greater than about 0.3 MHz and less than about 0.8 MHz; optionally greater than about 0.3 MHz and less than about 1 MHz; optionally greater than about 0.3 MHz and less than about 0.5 MHz; optionally greater than about 0.3 MHz and less than about 0.4 MHz; optionally greater than about 0.3 MHz and less than about 0.6 MHz; optionally greater than about 0.3 MHz and less than about 10 MHz; optionally greater than about 0.25 MHz and less than about 0.8 MHz; optionally greater than about 0.25 MHz and less than about 1 MHz; optionally greater than about 0.25 MHz and less than about 0.5 MHz; optionally greater than about 0.25 MHz and less than about 0.4 MHz; optionally greater than about 0.25 MHz and less than about 0.6 MHz; optionally greater than about 0.25 MHz and less than about 10 MHz; optionally greater than about 0.1 MHz and less than about 0.8 MHz; optionally greater than about 0.1 MHz and less than about 1 MHz; optionally greater than about 0.1 MHz and less than about 0.5 MHz; optionally greater than about 0.1 MHz and less than about 0.4 MHz; optionally greater than about 0.1 MHz and less than about 0.6 MHz; optionally greater than about 0.1 MHz and less than about 10 MHz; optionally greater than about 0.5 MHz and less than about 0.8 MHz; optionally greater than about 0.5 MHz and less than about 1 MHz; optionally greater than about 0.5 MHz and less than about 0.55 MHz; optionally greater than about 0.5 MHz and less than about 0.7 MHz; optionally greater than about 0.5 MHz and less than about 0.6 MHz; optionally greater than about 0.5 MHz and less than about 10 MHz; optionally greater than about 0.7 MHz and less than about 0.8 MHz; optionally greater than about 0.7 MHz and less than about 1 MHz; optionally greater than about 0.7 MHz and less than about 0.75 MHz; or optionally greater than about 0.5 MHz and less than about 10 MHz. Particularly advantageous acoustic frequencies are between about 0.3 MHz and about 0.7 MHz.

In ultrasound, acoustic intensity is a measure of power per unit of cross sectional area (e.g. mW/cm²) and requires averaging across space and time. The intensity of the acoustic beam can be quantified by several metrics that differ in the method for spatial and temporal averaging. These metrics are defined according to technical standards established by the American Institute for Ultrasound in Medicine and National Electronics Manufacturers Administration (NEMA. Acoustic Output Measurement Standard For Diagnostic Ultrasound Equipment (National Electrical Manufacturers Association, 2004)). A commonly used intensity index is the ‘spatial-peak, temporal-average’ intensity (Iₛₚₜₐ). The intensities reported herein refer to Iₛₚₜₐ at the targeted brain region. The spatial-peak temporal-average (Iₛₚₜₐ) intensity of the ultrasound wave in brain tissue is greater than about 0.0001 mW/cm² and less than about 1 W/cm², i.e. generally from 21 mW/cm² to 0.1 W/cm²; optionally from 21 mW/cm² to 0.5 W/cm²; optionally from 21 mW/cm² to 1 W/cm²; optionally from 50 mW/cm² to 0.1 W/cm²; optionally from 50 mW/cm² to 0.5 W/cm²; optionally from 50 mW/cm² to 1 W/cm²; optionally from 0.1 W/cm² to 0.2 W/cm²; optionally from 0.1 W/cm² to 0.5 W/cm²; and optionally from 0.1 W/cm² to 10 W/cm². Particularly advantageous Iₛₚₜₐ values are between about 100 mW/cm² and about 700 mW/cm², usually in the range from about 200 mW/cm² to about 500 mW/cm². The Iₛₚₜₐ value for any particular transcranial ultrasound neuromodulation protocol is calculated according to methods well known in the art that relate to the ultrasound pressure and temporal average of the transcranial ultrasound neuromodulation waveform over its duration. Effective ultrasound intensities for activating neurons or neuronal circuits do not cause tissue heating greater than about 2 degrees Celsius, usually less than 1 degree Celsius, for a period longer than about 5 seconds, preferably no longer than 3 seconds.

Significant attenuation of ultrasound intensity occurs at the boundaries between skin, skull, dura, and brain due to impedance mismatches, absorption, and reflection so the required ultrasound intensity delivered to the skin or skull may exceed the intensity at the targeted brain region by up to 10-fold or more depending on skull thickness and other tissue and anatomical properties.

Pulsing of ultrasound is an effective strategy for activating neurons that reduces the temporal average intensity while also achieving desired brain stimulation or neuromodulation effects. In addition to acoustic frequency and transducer variables, several waveform characteristics such as cycles per pulse, pulse repetition frequency, number of pulses, and pulse length affect the intensity characteristics and outcome of any particular transcranial ultrasound neuromodulation stimulus on brain activity. A pulsed transcranial ultrasound neuromodulation protocol generally uses pulse lengths between about 0.5 microseconds and about 1 second, i.e. generally from 0.5 microseconds to 5 microseconds; optionally from 0.5 microseconds to 50 microseconds; optionally from 0.5 microseconds to 100 microseconds; optionally from 0.5 microseconds to 500 microseconds; optionally from 0.5 microseconds to 1 ms; optionally from 0.5 microseconds to 10 ms; optionally from 0.5 microseconds to 100 ms; optionally from 0.5 microseconds to 500 ms; optionally from 0.5 microseconds to 1 second; optionally from 5 microseconds to 50 microseconds; optionally from 5 microseconds to 100 microseconds; optionally from 5 microseconds to 500 microseconds; optionally from 5 microseconds to 1 ms; optionally from 5 microseconds to 10 ms; optionally from 5 microseconds to 100 ms; optionally from 5 microseconds to 500 ms; optionally from 5 microseconds to 1 second; optionally from 100 microseconds to 500 microseconds; optionally from 100 microseconds to 1 ms; optionally from 100 microseconds to 10 ms; optionally from 100 microseconds to 100 ms; optionally from 100 microseconds to 500 ms; optionally from 100 microseconds to 1 second; optionally from 500 microseconds to 1 ms; optionally from 500 microseconds to 10 ms; optionally from 500 microseconds to 100 ms; optionally from 500 microseconds to 500 ms; optionally from 500 microseconds to 1 second; optionally from 1 ms to 10 ms; optionally from 1 ms to 100 ms; optionally from 1 ms to 500 ms; optionally from 1 ms to 1 second; and optionally from and 100 ms to 1 second. A transcranial ultrasound neuromodulation protocol may use pulse repetition frequencies (PRFs) between about 50 Hz and about 25 kHz, i.e. generally from 50 Hz to 100 Hz; optionally from 50 Hz to 250 Hz; optionally from 50 Hz to 1 kHz; optionally from 50 Hz to 2 kHz; optionally from 50 Hz to 3 kHz; optionally from 50 Hz to 4 kHz; optionally from 50 Hz to 5 kHz; optionally from 50 Hz to 10 kHz; optionally from 50 Hz to 25 kHz; optionally from 100 Hz to 250 Hz; optionally from 100 Hz to 1 kHz; optionally from 100 Hz to 2 kHz; optionally from 100 Hz to 3 kHz; optionally from 100 Hz to 4 kHz; optionally from 100 Hz to 5 kHz; optionally from 100 Hz to 10 kHz; optionally from 100 Hz to 25 kHz; optionally from 250 Hz to 500 Hz; optionally from 250 Hz to 1 kHz; optionally from 250 Hz to 2 kHz; optionally from 250 Hz to 3 kHz; optionally from 250 Hz to 4 kHz; optionally from 250 Hz to 5 kHz; optionally from 250 Hz to 10 kHz; optionally from 250 Hz to 25 kHz; optionally from 500 Hz to 1 kHz; optionally from 500 Hz to 2 kHz; optionally from 500 Hz to 3 kHz; optionally from 500 Hz to 4 kHz; optionally from 500 Hz to 5 kHz; optionally from 500 Hz to 10 kHz; optionally from 500 Hz to 25 kHz; optionally from 1 kHz to 2 kHz; optionally from 1 kHz to 3 kHz; optionally from 1 kHz to 4 kHz; optionally from 1 kHz to 5 kHz; optionally from 1 kHz to 10 kHz; optionally from 1 kHz to 25 kHz; optionally from 3 kHz to 4 kHz; optionally from 3 kHz to 5 kHz; optionally from 3 kHz to 10 kHz; optionally from 3 kHz to 25 kHz; optionally from 5 kHz to 10 kHz; optionally from 5 kHz to 25 kHz; and optionally from and 10 kHz to 25 kHz. Particularly advantageous PRFs are generally between about 1 kHz and about 3 kHz. For pulsed transcranial ultrasound neuromodulation waveforms, the number of cycles per pulse (cpp) is between about 5 and about 10,000,000. Particularly advantageous cpp values vary depending on the choice of other transcranial ultrasound neuromodulation parameters and are generally between about 10 and about 250. The number of pulses for pulsed transcranial ultrasound neuromodulation waveforms is between about 1 pulse and about 125,000 pulses. Particularly advantageous pulse numbers for pulsed transcranial ultrasound neuromodulation waveforms are between about 100 pulses and about 250 pulses.

Tone bursts of ultrasound energy that extend for about 1 second or longer—though, strictly speaking, also pulses—are often referred to as continuous wave (CW). In alternative embodiments, one or more continuous wave (CW) ultrasound waveforms less than about five seconds in duration, typically from 1 second to 5 seconds. US protocols that include such CW waveforms offer advantages for neuromodulation due to their capacity to drive activity robustly. However, one disadvantage of transcranial ultrasound neuromodulation protocols with CW pulses is that the temporal average intensity is significantly higher which may cause painful thermal stimuli on the scalp or skull and may also induce heating and thus damage in brain tissue. Thus, advantageous embodiments using CW pulses may employ a lower acoustic intensity and/or a slow pulse repetition frequency of less than about 1 Hz. For instance, a CW US stimulus waveform with 1 second pulse lengths repeated at 0.5 Hz would deliver US every other second. Alternative pulsing protocols including those with slower pulse repetition frequencies of less than about 0.5 Hz or less than about 0.1 Hz or less than about 0.01 Hz or less than about 0.001 Hz are also beneficial. In some useful embodiments, the interval between pulses or pulse length may be varied during a transcranial ultrasound neuromodulation protocol that includes CW pulses.

Providing a mixture of ultrasound frequencies is useful for efficient brain stimulation. Various strategies for achieving a mixture of ultrasound frequencies to the brain of the user are known. A strategy for producing ultrasound waves that contain power in a range of frequencies is to use square waves to drive the transducer or drive the transducer off-resonance. Another strategy for generating a mixture of ultrasound frequencies is to choose transducers that have different center frequencies and drive each at their resonant frequency. One or more of the above strategies or alternative strategies known to those skilled in the art for generating US waves with a mixture of frequencies would also be beneficial. Mixing, amplitude modulation, or other strategies for generating more complex transcranial ultrasound neuromodulation waveforms can be beneficial for driving distinct brain wave activity patterns or to bias the power, phase, or spatial extent of brain oscillations such as slow-wave, delta, beta, theta, gamma, or alpha rhythms.

In some embodiments, the transcranial ultrasound neuromodulation system or device is configured to target one or more regions of cerebral cortex, where the region of cerebral cortex chosen from the group that includes, but is not limited to the: striate visual cortex, visual association cortex, primary and secondary auditory cortex, somatosensory cortex, primary motor cortex, supplementary motor cortex, premotor cortex, the frontal eye fields, prefrontal cortex, orbitofrontal cortex, dorsolateral prefrontal cortex, ventrolateral prefrontal cortex, anterior cingulate cortex, and other area of cerebral cortex.

A transcranial ultrasound neuromodulation system or device as described herein may be configured to target one or more deep brain regions chosen from the group that includes, but is not limited to: the limbic system (including the amygdala), hippocampus, parahippocampal formation, entorhinal cortex, subiculum, thalamus, hypothalamus, white matter tracts, brainstem nuclei, cerebellum, neuromodulatory nucleus, or other deep brain region.

In some embodiments, the transcranial ultrasound neuromodulation system or device is configured to target one or more brain regions that mediate sensory experience, motor performance, and the formation of ideas and thoughts, as well as states of being chosen from the group that includes, but is not limited to: emotion, physiological arousal, sexual arousal, attention, creativity, relaxation, empathy, connectedness, motivation, and other cognitive states.

In some embodiments, the transcranial ultrasound neuromodulation system or device is configured to modulate neuronal activity underlying multiple sensory domains and/or cognitive states occurring concurrently or in close temporal arrangements.

In some embodiments, the effect of delivering ultrasound energy to one or more brain regions is a modulation of one or a plurality of biophysical or biochemical processes chosen from the group that includes, but is not limited to: (i) ion channel activity, (ii) ion transporter activity, (iii) secretion of signaling molecules, (iv) proliferation of the cells, (v) differentiation of the cells, (vi) protein transcription of cells, (vii) protein translation of cells, (viii) protein phosphorylation of the cells, and (ix) protein structures in the cells.

### Example 1

### Focused Ultrasound (FUS) Waveform Generation

In an exemplary embodiment, transcranial ultrasonic neuromodulation waveforms may be generated using a two-channel, 2 MHz function generator (e.g., BK Precision Instruments) Channel one is set to deliver ultrasound at a pulse repetition frequency (PRF) of 1.0 kHz and channel two is set to drive the transducer at a 0.5 MHz acoustic frequency (Af) in a bursting mode with channel one serving as an external trigger for channel two. The pulse duration (PD) of the waveform may be set to 0.36 msec by adjusting the number of cycles per pulse (c/p) on channel two to 180 while the stimulus duration (0.5 sec) is set by adjusting the number of pulses (np) on channel one to 500. The output of channel two is sent through a 40 W linear RF amplifier (e.g., E&I 240L; Electronics & Innovation) before being sent to a custom designed focused ultrasound transducer (e.g., Blatek, College Station Pa.) having a center frequency (fc) of 0.5 MHz, a diameter (d) of 30 mm, and a focal length (F) of 30 mm. The waveform employed for transcranial focused ultrasound (tFUS) stimulation has the following parameters: A_{f}=0.50 MHz, PD=360 μsec, PRF=1.0 kHz and np=500 to produce a stimulus duration of 0.5 sec yielding a peak rarefactional pressure (pr) of 0.80 MPa, a mechanical index (MI) of 1.13, and a spatial-peak pulse-average intensity (I_{SPPA}) of 23.87 W/cm² prior to transcutaneous and transcranial transmission. This waveform does not produce heating of the skin or skull bone. The transducer may be coated with acoustic coupling gel and placed on the scalp at the 10-20 electrode location CP3 before being secured in place (e.g., via an athletic pre-wrap bandaging).

### Example 2

### Modeling of Acoustic Pressure Fields in the Brain with Finite Element Method Simulations

To gain insight regarding the intracranial spatial patterns and resolution of US induced pressure waves, a simple model was constructed using the finite element method (FEM) with COMSOL Multiphysics software (COMSOL, Inc., Burlington, Mass.). The modeling domain consisted of a circle (r=9 cm) to approximate the brain encompassed by a 5 mm thick annulus representing the skull, and a larger annulus (r=15 mm) outside the skull to provide an outer boundary of skin and acoustic coupling gel. This simple 2D geometry approximates the head as an infinite cylinder that is valuable for developing an understanding of the basic insertion behavior of US as it propagates across several model tissue layers (skin and skull) into the brain. The density (p) of brain was specified as 1,030 kg/m³ and the speed of sound (c) was 1550 m/sec. For the skull, p was set to 1,912 kg/m³ and c was estimated as 2,300 m/sec based on previous empirical observations. The outermost annulus for skin and ultrasound gel was specified to have the material properties of water. A plane wave incident pressure field of 100 Pa from the negative axial direction was used to represent stimulation from the US transducer.

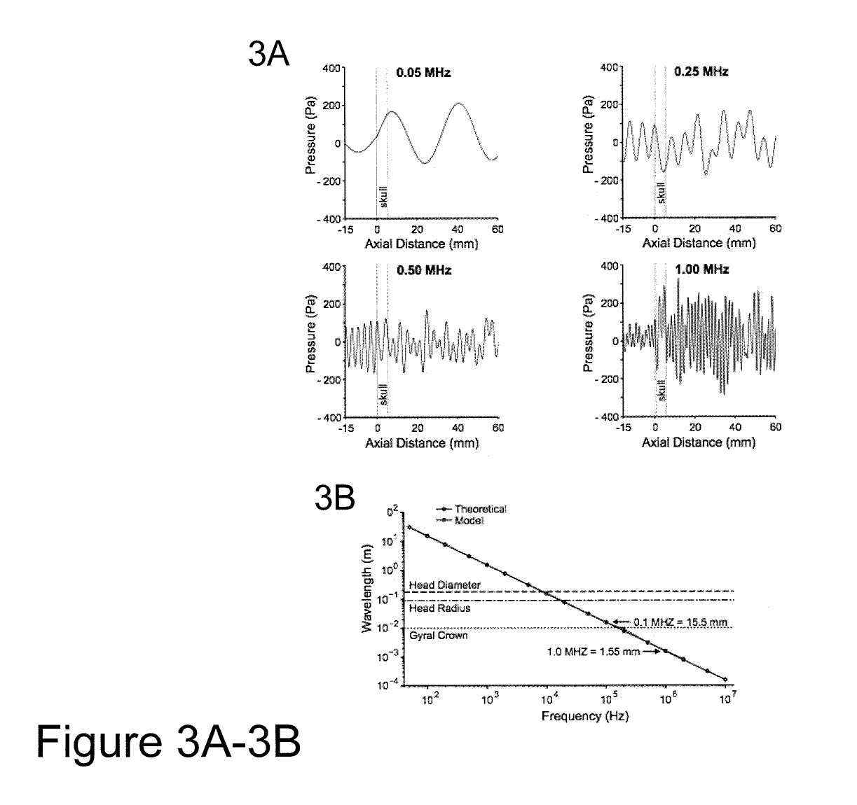

The length of elements in the mesh used for solution of any FEM model plays a crucial role in the correctness of the obtained solution, as well as the computational cost of simulations. For these simple models, a smaller mesh size (1 mm) was used for analysis of the resultant sound pressure field. The pressure profile is extracted along a radius perpendicular to the planar acoustic waves in the FEM to model the intracranial wavelength of US (FIGS. 3A-3B). To calculate the spatial resolution for a particular US frequency, the average distance between the peaks of the pressure profiles were measured as they propagated across the model tissue layers. Assuming a linear homogenous media, the theoretical resolution can be calculated as the wavelength (λ) of the sinusoidal waveform, which is dependent on the speed of sound in the material and the wave frequency (f), by λ=c/f.

Using these methods, an FEM simulation of the human head may be used to facilitate a general understanding of the spatial resolution of intracranial sound pressure waves as a function of acoustic frequency. Using this model simulation of the pressure profile along the central axis of longitudinal sound waves along a plane perpendicular to a transducer across a range of acoustic frequencies is possible. Following distortion of the pressure wave transmitted through the skull (located at axial distance zero), acoustic waves continue to propagate into and through the brain having a wavelength dependent on the frequency of the incident wave, as shown in FIG. 3A for different frequencies/wavelengths. With increasing frequency, the wavelength of the intracranial sound pressure decreases yielding increased spatial resolutions. The sound pressure wavelengths in the brain following transcranial transmission estimated using FEM simulations are shown in comparison to theoretical sound pressure wavelengths in brain tissue for various frequencies in FIG. 3B. The wavelengths simulated using FEM models are in close agreement with theoretical predictions. In this embodiment, acoustic frequencies below 0.01 MHz yield spatial resolutions greater than the diameter of the head, while a US frequency of about 0.1 MHz is necessary to obtain a lateral spatial resolution of approximately 1 cm, which is a length scale approximating the size of a human gyral crown. The FEM model predicts a lateral spatial resolution of approximately 3.1 mm for 0.5 MHz US in brain tissue following transcranial transmission (FIG. 3).

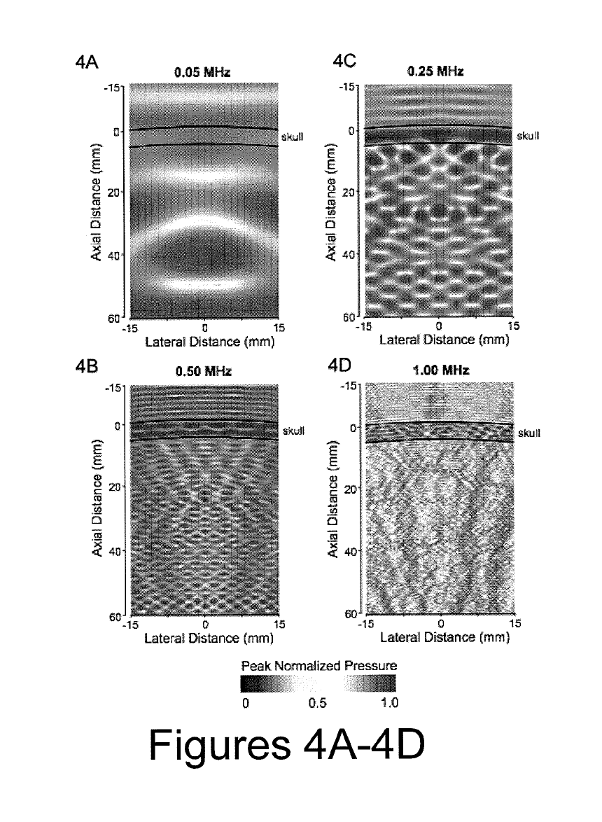

Due to the mismatch of material properties (for example, density) between the skull and brain, incident sound pressure waves refract as they are transmitted across these layers. This bending of sound waves can be further exacerbated by the curvature of the material interfaces (skin, skull, and cerebrospinal fluid space) to produce a slight focusing effect on a planar acoustic pressure field, even without an acoustic lens, as shown in FIGS. 4A-4D. The modeled diffraction patterns for transcutaneous and transcranial planar US in brain are illustrated for different acoustic frequencies ranging from 0.05 (FIG. 4A) to 1.0 MHz (FIG. 4D). Analogous to the pressure profiles in illustrated in FIG. 3A sound pressure nodes become denser in the modeled brain to effectively increase the spatial resolution of transcranial planar US as acoustic frequency increases.

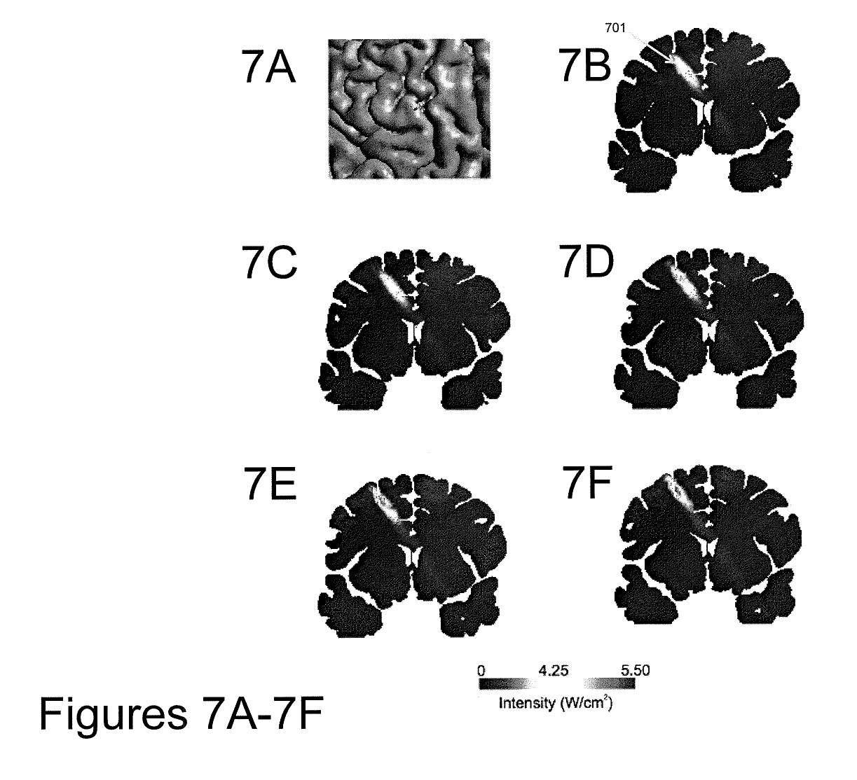

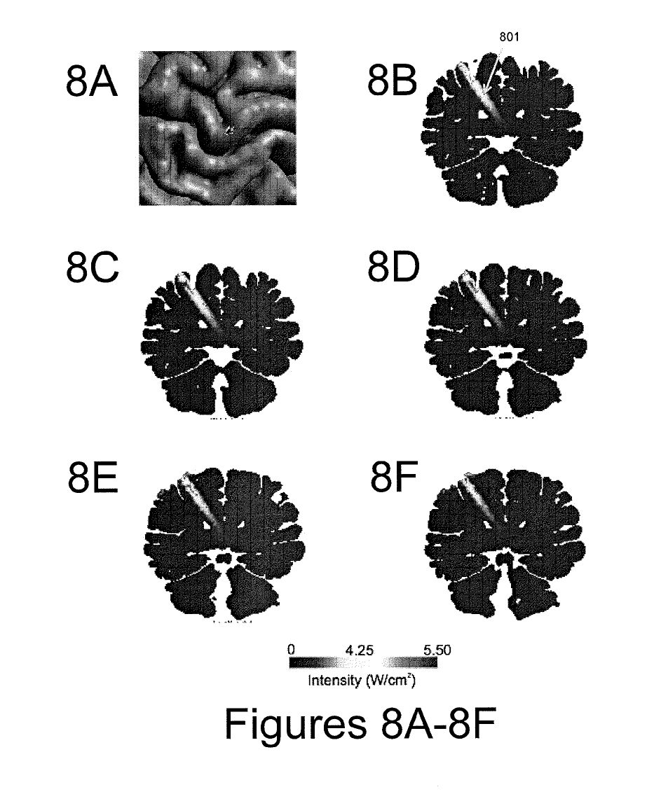

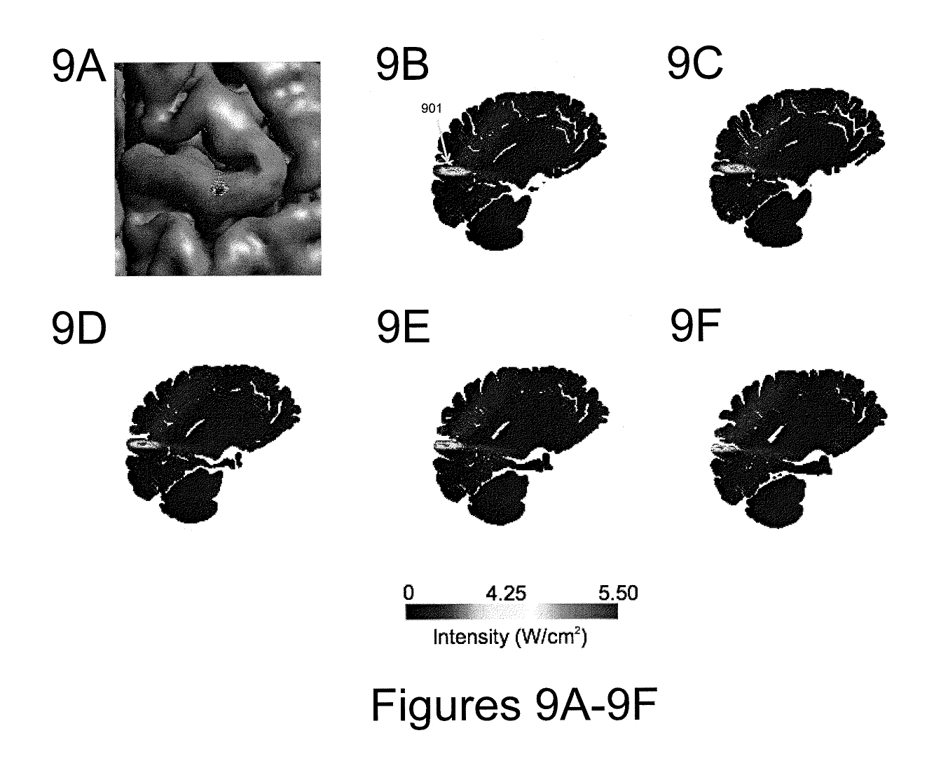

FIGS. 7A-7F, 8A-AF and 9A-9F show overlays of the experimentally measured field maps as shown in measurements plotted in FIGS. 3A-3B, 4A-4D, 5A-5D and 6A-6D onto an anatomical model of the brain. Top-down and cut-away views show the acoustic field of transcranial focused ultrasound being projected into a realistic anatomical model of the brain, which was derived from whole head structural MR images. Projection of the transcranial focused ultrasound acoustic field clearly illustrates the targeting of dorsolateral prefrontal cortex 701 (FIGS. 7A-7F), primary motor cortex 801 (FIGS. 8A-8F), and occipital cortex 901 (FIGS. 9A-9F).

### Example 3

### Quantitative Acoustic Field Mapping

Measurements of an acoustic intensity profile of the waveform may be done using a calibrated hydrophone (e.g., HNR-0500, ONDA Corporation, Sunnyvale Calif.) whose signal was amplified by an AH-1100 preamplifier (e.g., Onda Corporation). The hydrophone, US transducer, and skull fragment may be positioned within an acrylic tank (e.g., 15 gallon acrylic tank). The hydrophone may be mounted on a three-axis stage (e.g., LTS300, Thorlabs Inc, Newton N.J.) using an assortment of optomechanical components (e.g., Edmund Optics Inc., Barrington, N.J. and Thorlabs Inc., Newton, N.J.). The US transducer and skull fragment may be similarly positioned. Custom software may be utilized to control the three-axis stage as well as the timing of transducer excitation and recording of the corresponding waveform as measured by the hydrophone. Acoustic field scans can be performed at spatial intervals, for instance, 400 μm (2 to 122 mm away from transducer in a 10.4 mm×10.44 mm region) and 200 μm (2 to 72 mm away from transducer in a 5.6 mm×5.6 mm region). For finding the final focal plane as well as the spatial peak location, field map(s) obtained from the earlier scans may be used as locators for conducting 100 μm resolution scans. Scans around the axis (Z axis) can be first performed to find the focal distance; next, a 12 mm×12 mm scan can be performed at this distance to obtain an XY acoustic power map at the focal plane. Scans can be first performed without the skull in between the transducer and hydrophone. Subsequently, to test the effects of a human skull on FUS fields, a 6 mm thick fragment of human cortical bone (rehydrated for 48 hours) may be inserted in between the transducer and the hydrophone and scans repeated using the same procedures, except that the starting distance to the transducer was increased to 10 mm to avoid colliding the skull and hydrophone.

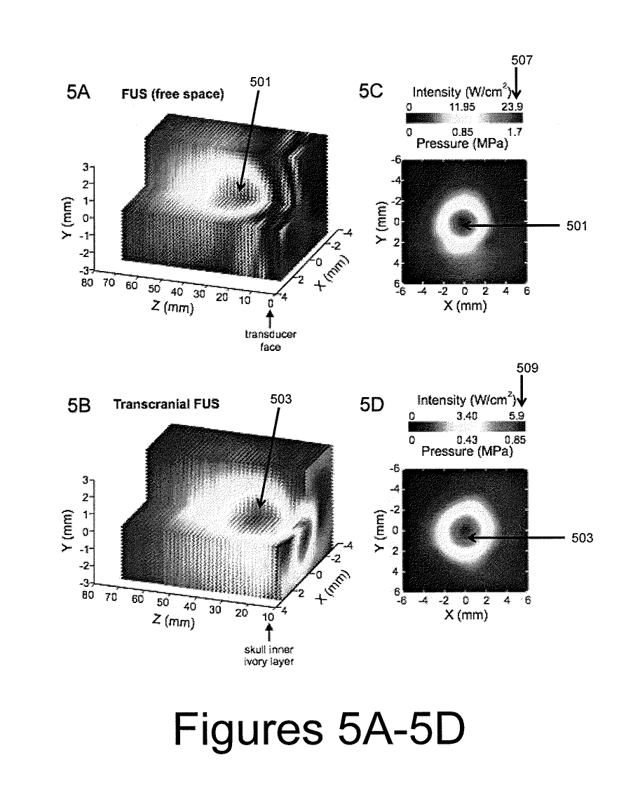

FIGS. 5A-5D show acoustic fields emitted from a 0.5 MHz FUS transducer as measured in free space (FIG. 5A; no skull) and following transmission through a hydrated human cranium fragment (FIG. 5B; transcranial focused ultrasound). The gradient look-up table (FIG. 5C, top) shows the acoustic intensity and peak-to-peak pressure for each experimental condition. Arrows 501, 503 show the regions of highest intensity (corresponding to the region indicated by arrows 507, 509, respectively in FIGS. 5C, top and 5D, top) The three-dimensional acoustic field maps (FIGS. 5C and 5D) may be measured using a calibrated hydrophone scanning through an acoustic test tank in 200 μm increments. Cross-sections of the focal planes illustrate the lateral (XY) resolutions of the 0.5 MHz focused ultrasound pressure fields (right) for both the free space (no skull) and transcranial conditions.

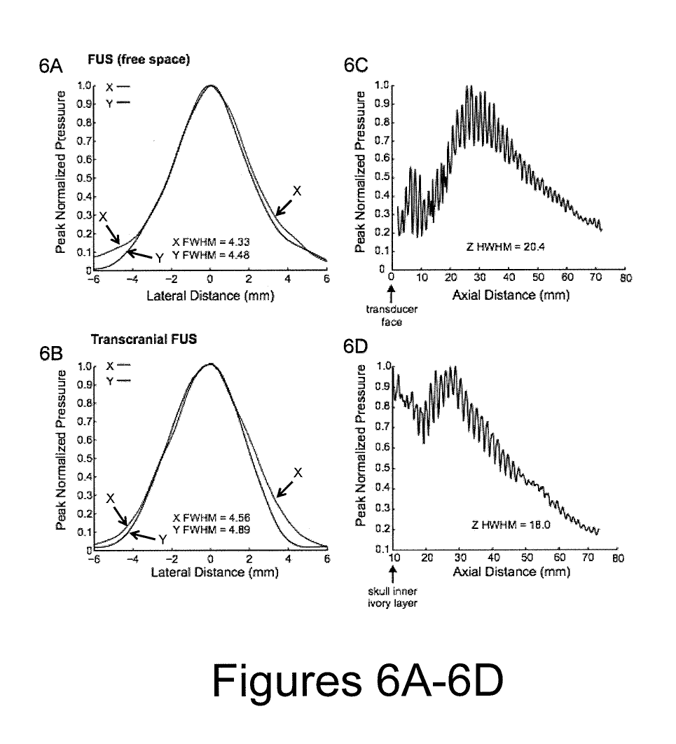

FIGS. 6A and 6B show line plots illustrate the peak normalized pressure profiles and show the X (arrow labeled “X”) and Y (arrow labeled “Y”) lateral resolutions in the focal plane for 0.5 MHz FUS fields transmitted into free space (FIG. 6A; no skull) and through a human cranium fragment (FIG. 6B). Also illustrated are line plots showing the axial (Z) pressure profiles (right) of the FUS fields for both the free space (FIG. 6A) and transcranial (FIG. 6B) conditions. Note the acoustic intensity in the axial direction drops off faster after being transmitted through the skull bone compared to the free space condition indicating a reduced depth-of-field, whereas the lateral extent of the pressure profile is similar for the no skull and transcranial conditions.

### Example 4

### Quantitative Analysis of the Effect of the Skull on Compound Convex-Concave Focused Ultrasound

Using a calibrated hydrophone mounted on a motorized three-axis stage it is possible to record acoustic pressure fields of 0.5 MHz FUS transmitted into the free space of an acoustic test tank, as well as through hydrated fragments of human cranium bone. Recording these acoustic pressure fields provides insights for resolving the following three issues. The first is the characterization of the loss of acoustic power due to transmitting US across human skull bone. The second is the characterization of the three-dimensional spatial resolution of transcranial FUS (tFUS) emitted from a single element transducer. Related to the spatial resolution, the third objective is the study of the effect of human skull on the shape of tightly focused acoustic fields. Measurements reveal that the spatial-peak pulse-average intensity (I_{SPPA}) drops by approximately four-fold using skull samples (1/4.05, corresponding to a −6.07 dB insertion loss). Intensity drop-off may be measured across a range of acoustic powers (free space powers and pressures ranging from I_{SPPA}=0.12 W/cm² and 0.12 MPa peak-to-peak pressure to I_{SPPA}=50 W/cm² and 2.5 MPa peak-to-peak pressure respectively) and ranged from a 3.7 to a 4.1 fold drop (data not shown), with a slight trend towards increasing losses at higher powers. One cause of this may be nonlinearities in the system.

While analyzing the acoustic field shape, it is desirable to consider 50% and 20% of the pressure maximum as our spatial targets. Compared to FUS transmitted into free space, transmitting FUS through the skull causes an approximately 10% loss in the lateral spatial resolution as estimated by the full width at half maximum (FWHM; FIGS. 6A-6B). When measuring the width at the 20% pressure maximum however, the tFUS field is slightly more focused than the FUS field measured in free space. The FUS pressure half width of the half maximum (HWHM) is 20.4 mm in the free space condition and 18.0 mm following transcranial transmission (FIGS. 6C-6D). Under these conditions, transmission of 0.5 MHz FUS through the skull may lead to a reduced pressure depth-of-field and an approximately 12% increase in the axial resolution. This natural focusing may be best described where nonlinear effects cause a cone of FUS to rotate back towards skull insertion point creating a more compact pressure field corresponding to reduced brain tissue penetration depth (FIGS. 4A-4D and 6B). As discussed above the effects of transcranial transmission also has mild effects on lateral resolution depending on the pressure drop measured. In sum, the skull is not necessarily an obstacle for transcranial focusing of US and may actually enhance it under certain conditions.

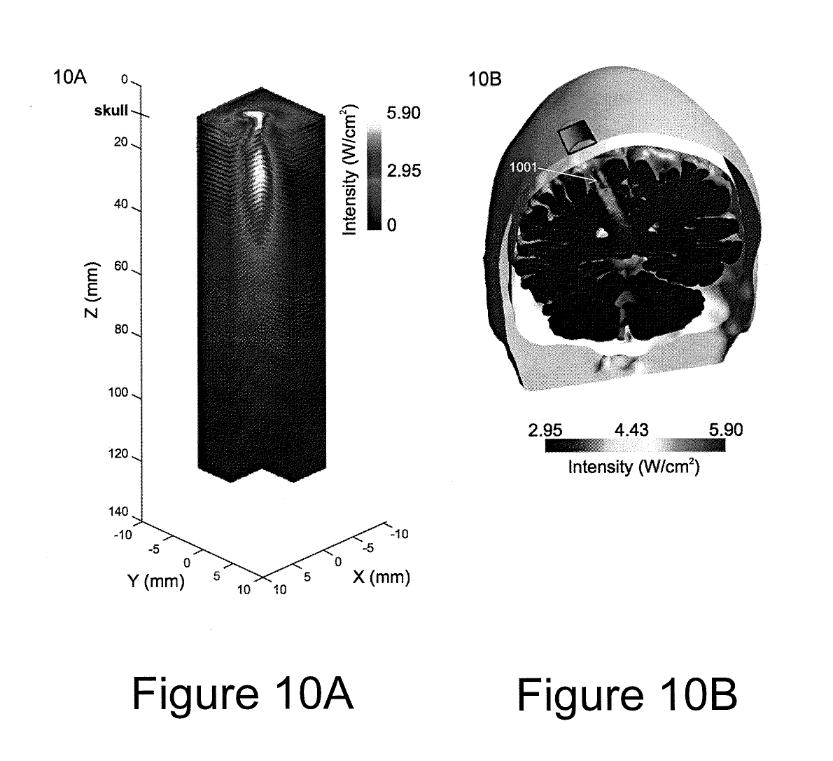

Another example of the application of the acoustic energy focused to have a short focal length at therapeutically relevant intensities, dimensions and locations is shown in FIGS. 10A and 10B. FIG. 10A shows intensity measured with a hydrophone for acoustic waves generated by an ultrasound transducer and transmitted through a compound (“Vexcave”) acoustic lens as described herein. In this example, the compound acoustic lens has a focal distance of 25 mm, sufficient to pass the sound through skull bone to reach cortical brain regions. In FIG. 10A, the face of the transducer is at 0 mm (outside of the head). The peak intensity is at approximately 25 mm from the transducer (approximately 15 mm into the head (e.g., from the inner edge of the skull)), appropriate for cortical stimulation.

FIG. 10B projects the measured acoustic intensity from FIG. 10A onto an anatomically realistic finite element model (FEM) of a human head, including skull, dura, and brain, showing how the peak intensity (arrow 1001) is located at a cortical brain region.

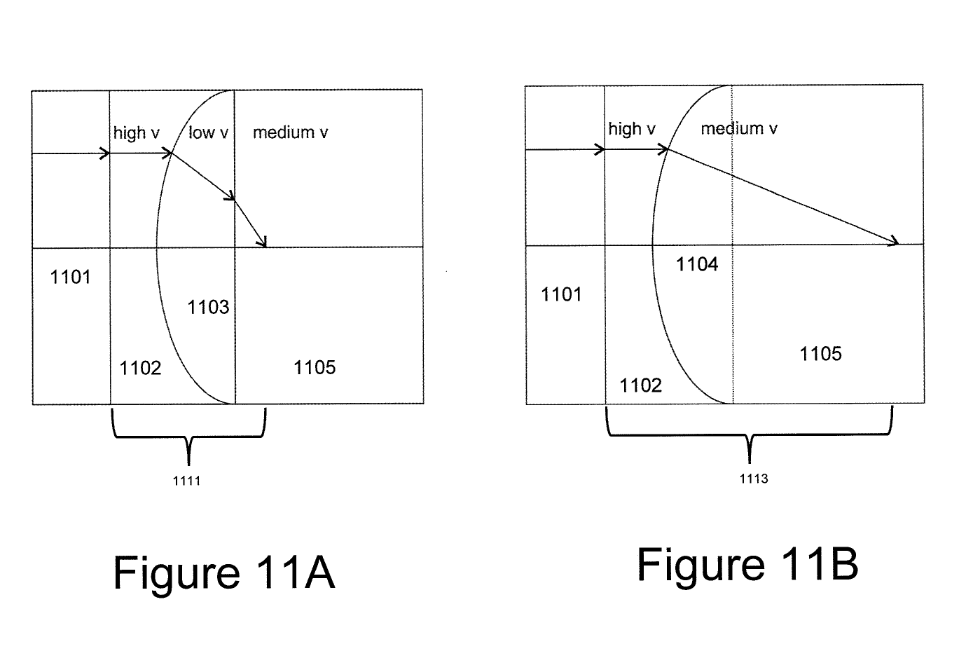

FIGS. 11A and 11B illustrate using ray diagrams roughly how the compound acoustic lenses described above (shown schematically in FIG. 11A) differ from traditional single-lens systems, such as a focusing concave lens (shown schematically in FIG. 11B). A compound Vexcave acoustic lens assembly can achieve a shorter focal length 1111 than a single-lens system 1113. Both schematics include ultrasound transducer 1101, plano-concave lens 1102 comprised of a material having a high acoustic velocity (‘high v’, e.g., higher than the acoustic velocity of water), and target tissue 1105 having an acoustic velocity similar to water (‘medium v’). In this schematic, the tissue 1105 may also include an ultrasound gel or other ultrasound coupling component. The arrows illustrate an approximate path of acoustic energy emitted from the transducer and focused through the compound acoustic lens. FIG. 11A shows an assembly that has an additional plano-convex lens 1103 comprised of a material having a low acoustic velocity (‘low v’) in accordance with embodiments of the invention. FIG. 11B shows an assembly that is not a compound lens, wherein area 1104 may comprise an ultrasound gel or other matching layer to target tissue 1105.

The large difference in acoustic velocity between plano-concave lens 1102 and plano-convex lens 1103 in this example causes sharper focusing of acoustic energy. Moreover, the difference in acoustic velocity between plano-convex lens 1103 and tissue & ultrasound couplant 1105 causes additional focusing to achieve a closer focal point than occurs with a single plano-concave lens in FIG. 11B.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements, these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range recited herein is intended to include all sub-ranges subsumed therein.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A compound acoustic lens apparatus having a short focal length for use with a transcranial ultrasound system, the apparatus comprising:
an ultrasound transducer having a diameter;
a concave lens coupled to the ultrasound transducer, wherein the concave lens has an acoustic velocity that is greater than an acoustic velocity of water; and
a convex lens coupled to the concave lens, wherein the convex lens has an acoustic velocity that is less than the acoustic velocity of water;
further wherein the focal length of the compound acoustic lens is 1.5 times the diameter of the ultrasound transducer or less when a frequency of acoustic energy applied from the compound acoustic lens is between about 0.2 MHz and 1 MHz at a spatial-peak, temporal-average intensity of about 10 watts/cm² or less.

2. A compound acoustic lens apparatus having a short focal length for use with a transcranial ultrasound system, the apparatus comprising:
an ultrasound transducer having a diameter of about 15 mm or greater;
a concave lens coupled to the ultrasound transducer, wherein the concave lens has an acoustic velocity that is greater than an acoustic velocity of water; and
a convex lens coupled to the concave lens, wherein the convex lens has an acoustic velocity that is less than the acoustic velocity of water;
wherein the focal length of the compound acoustic lens is less than twice the diameter of the ultrasound transducer when a frequency of acoustic energy applied through the compound acoustic lens is between about 0.2 MHz and 1 MHz at a spatial-peak, temporal-average intensity of about 10 watts/cm² or less.

3. The apparatus of claim 1, wherein the diameter of the ultrasound transducer is 15 mm or greater.

4. The apparatus of claim 1, wherein the focal length of the compound acoustic lens is 1.5 times the diameter of the ultrasound transducer or less when a frequency of acoustic energy applied from the compound acoustic lens is between about 0.2 MHz and 1 MHz at a spatial-peak, temporal-average intensity of between about 0.5 and about 10 watts/cm² at a target brain region.

5. The apparatus of claim 1, wherein the concave lens is immediately adjacent a face of the ultrasound transducer and the convex lens is immediately adjacent the concave lens.

6. The apparatus of claim 1, wherein the convex lens is a plano-convex lens having a convex surface facing away from the transducer.

7. The apparatus of claim 1, wherein the concave lens is a plano-concave lens having a concave surface facing the transducer.

8. The apparatus of claim 1, wherein an acoustic impedance of the transducer is greater than an acoustic impedance of the concave lens, and the acoustic impedance of the concave lens is greater than an acoustic impedance of the convex lens.

9. The apparatus of claim 1, wherein the concave lens comprises a focal length of less than 35 mm.

10. The apparatus of claim 1, wherein the concave lens comprises a material selected from the group consisting of: graphite or aluminum.

11. The apparatus of claim 1, wherein the convex lens comprises a material selected from the group consisting of: silicone rubbers, balsa wood, and cork.

12. The apparatus of claim 2, wherein the focal length of the compound acoustic lens is 1.5 times the diameter of the ultrasound transducer or less.

13. A system for neuromodulation by transcranial ultrasound, the system comprising:
an ultrasound transducer having a diameter;
a compound acoustic lens having a short focal length, the apparatus comprising:
a concave lens coupled to the ultrasound transducer, wherein the concave lens has an acoustic velocity that is greater than an acoustic velocity of water, and
a convex lens coupled to the concave lens, wherein the convex lens has an acoustic velocity that is less than the acoustic velocity of water,
wherein the focal length of the compound acoustic lens is less than 1.5 times the diameter of the ultrasound transducer during operation of the system; and
a driver coupled to the ultrasound transducer and configured to drive the ultrasound transducer to emit a frequency of acoustic energy from the compound acoustic lens between about 0.2 MHz and 1 MHz at a spatial-peak, temporal-average intensity of about 10 watts/cm² or less.

14. The system of claim 13, wherein the diameter of the ultrasound transducer is 15 mm or greater.

15. The system of claim 13, wherein the concave lens is immediately adjacent a face of the ultrasound transducer and the convex lens is immediately adjacent the concave lens.

16. The system of claim 13, wherein the convex lens is a plan-convex lens having a convex surface facing the transducer.

17. The system of claim 13, wherein the concave lens is a plano-concave lens having a concave surface facing away from the transducer.

18. The system of claim 13, wherein an acoustic impedance of the transducer is greater than an acoustic impedance of the concave lens, and the acoustic impedance the concave lens is greater than an acoustic impedance of the convex lens.

19. The system of claim 13, wherein the concave lens comprises a focal length of less than 35 mm.

20. The system of claim 13, wherein the concave lens comprises a material selected from the group consisting of: graphite or aluminum.

21. The system of claim 13, wherein the convex lens comprises a material selected from the group consisting of: silicone rubbers, balsa wood, and cork.

22. The system of claim 13, wherein the driver is configured to drive the ultrasound transducer to emit a frequency of acoustic energy from the compound acoustic lens between about 0.2 MHz and 1 MHz at a spatial-peak, temporal-average intensity of between about 0.5 and about 10 watts/cm² at a target brain region.

23. A method for transcranial neuromodulation by applying transcranial ultrasound using a compound acoustic lens having a short focal length, the method comprising:
driving an ultrasound transducer having a diameter, to emit a frequency of acoustic energy from between about 0.2 MHz and 1 MHz;
passing the acoustic energy through a concave lens of a compound acoustic lens that is attached to the ultrasound transducer, wherein the concave lens has an acoustic velocity that is greater than an acoustic velocity of water;
passing the acoustic energy from the concave lens through a convex lens of the compound acoustic lens, wherein the convex acoustic lens has an acoustic velocity that is less than the acoustic velocity of water; and
focusing the acoustic energy leaving the compound acoustic lens at a focal length of less than 1.5 times the diameter of the ultrasound transducer to target a brain region and deliver the acoustic energy at a spatial-peak, temporal-average intensity of about 10 watts/cm² or less to the target brain region.

24. The method of claim 23, wherein focusing comprises non-invasively focusing the acoustic energy on a cortical region of a brain.

25. The method of claim 23, further comprising positioning the ultrasound transducer and compound acoustic lens against a subject's head.

26. The method of claim 23, further comprising positioning the ultrasound transducer and the compound acoustic lens against a solid ultrasound couplant placed against a subject's head.

27. The method of claim 23, further comprising positioning the ultrasound transducer and the compound acoustic lens against an ultrasound couplant comprising silicone that is placed against a subject's head.

28. The method of claim 23, wherein driving an ultrasound transducer comprises driving the ultrasound transducer having the diameter of 15 mm or greater.

29. The method of claim 23, wherein passing the acoustic energy through the concave lens comprises passing the acoustic energy through the concave lens that is immediately adjacent a face of the ultrasound transducer on one side and is immediately adjacent the convex lens on the opposite side.

30. The method of claim 23, wherein passing the acoustic energy from the concave lens through a convex lens comprises passing the acoustic energy from the convex lens wherein the convex lens is a plano-convex lens having a convex surface facing the transducer.

31. The method of claim 23, wherein passing the acoustic energy through the concave lens comprises passing the acoustic energy through the concave lens wherein the concave lens is a plano-concave lens having a concave surface facing away from the transducer.

32. The method of claim 23, wherein focusing the acoustic energy leaving the compound acoustic lens comprises focusing the acoustic energy at a focal length of less than 35 mm.
